(19) United States
(12) Patent Application Publication
(10) Pub. No.: US 2012/0323229 A1
(43) Pub. Date: Dec. 20, 2012

(54) EYE-SAFE DEVICE FOR TREATMENT OF SKIN TISSUE

(75) Inventors: Henry Zenzie, Dover, MA (US); Gregory B. Altshuler, Lincoln, MA (US); Oldrich M. Laznicka, Wellesley, MA (US)

(73) Assignee: PALOMAR MEDICAL TECHNOLOGIES, INC., Burlington, MA (US)

(21) Appl. No.: 13/600,607

(22) Filed: Aug. 31, 2012

Related U.S. Application Data
(62) Division of application No. 12/143,520, filed on Jun. 20, 2008.
(60) Provisional application No. 60/936,575, filed on Jun. 21, 2007.

(51) Int. Cl. A61B 18/18; A61N 5/06

(52) U.S. Cl. 606/9; 607/88

(57) ABSTRACT

Methods and devices are described that provide for eye-safe treatments using a photocosmetic device on skin tissue. In particular, various eye-safety devices protect the eye, including both the retina and the iris, as well as provide additional skin safety. The devices and methods described are particularly useful in consumer devices, but are useful in other devices also.

### RELATED APPLICATIONS

This application claims the benefit of U.S. Provisional Application No. 60/936,575 filed Jun. 21, 2007, which is incorporated herein by reference in its entirety.

### BACKGROUND OF THE INVENTION

1. Technical Field

Eye-safe methods and apparatus for utilizing electromagnetic radiation to treat cosmetic and health conditions are described, including, without limitation, methods and apparatus for treating soft tissue using a suitable handheld device operated by a consumer.

2. Background

There exists a variety of conditions treatable using photocosmetic procedures (also referred to herein as photocosmetic treatments), including light-based (e.g., using a laser, lamp or other light source) hair growth management, treatment of pseudofolliculitis barbae, treatment of acne, treatment of various skin lesions (including pigmented and vascular lesions), leg vein removal, tattoo removal, facial resurfacing, treatment of fat, including cellulite, removal of warts and scars, and skin rejuvenation, including treatment of wrinkles and improving skin tone and texture, and various other dermatologic, cosmetic and other treatments.

Currently, various photocosmetic procedures are performed using professional-grade devices that cause destructive heating of target structures located in the epidermis or dermis of a patient's skin. These procedures are typically performed in a physician's office or the office of another licensed practitioner, partially because of the expense of the devices used to perform the procedures, partially because of safety concerns related to the devices, and partially because of the need to care for optically induced wounds on the patient's skin. Such wounds may arise from damage to a patient's epidermis caused by the high-power radiation and may result in significant pain and/or risk of infection.

While certain photocosmetic procedures, such as CO₂ laser facial resurfacing, will continue to be performed in the dermatologist's office for medical reasons (e.g., the need for post-operative wound care), there are a large number of photocosmetic procedures that could be performed in either a medical or in a non-medical environment (e.g., home, barber shop, or spa), if the consumer could perform the procedure in a safe and effective manner. Even for procedures performed in a medical environment, less expensive, safer and easier to use devices would be advantageous and reduced skin damage would reduce recovery time.

Photocosmetic devices for use in medical or non-medical environments preferably should be designed to be safe for use on the skin or other tissues, and, for example, to prevent eye and skin injuries, including damage to a patient's iris even when an eye lid is closed. Such devices also preferably should be designed to be easy to use, thus allowing an operator to achieve acceptable cosmetic results with only simple instructions and potentially to enhance the overall safety of the device. The safety of currently available photocosmetic devices, including those used in the professional setting, could be improved in these areas.

For example, eye-safe consumer devices would prevent accidental injuries to users of those devices. Prior art solutions to provide eye safety generally have been directed to protecting the retina and may not protect a patient's iris. The iris often includes a high concentration of melanin which may absorb treatment energy even when the eye lid is closed. Often eye protection techniques (e.g. frosted glass, defocused optics, low power) negatively impact the efficacy of treatment.

Existing consumer devices sold to consumers are generally of very low power and are only marginally effective, at best. The safety measures on such devices may not adequately protect the retina, iris or any other part of the eye or other tissue when used in conjunction with a consumer device designed to irradiate tissue using higher power densities and fluences. Therefore, it is desirable to provide a skin treatment device which provides increased eye safety, to allow a consumer or other user to safely use the device at higher powers and levels of efficacy than have been developed to this point.

### SUMMARY OF THE INVENTION

Eye-safe methods and apparatus for treating soft tissue with electromagnetic radiation (EMR) using a suitable handheld device operated by a consumer are described. Although most embodiments are described with respect to using a handheld consumer device with a laser providing the source of EMR, many of the principles described herein are also applicable to other embodiments, including, without limitation, devices operated by professionals and devices using lamps or other sources of EMR.

One aspect of the invention is an apparatus for photocosmetic treatments that has a housing with an aperture and a source capable of producing electromagnetic radiation. The source emits the electromagnetic radiation through the aperture. The apparatus also has at least one pressure sensor adjacent to the aperture. The pressure sensor is configured to actuate the source when pressure on the sensor exceeds a predetermined threshold. The threshold is configured to prevent the source from being actuated when the aperture is placed against eye tissue.

Preferred embodiments of this aspect of the invention may include some of the following additional features. The pressure sensor may have a set of pins located about the perimeter of the aperture. The housing may have a movable head in mechanical communication with the pressure sensor. The housing may have a reflectometer or a pigmentometer.

Another aspect of the invention is an apparatus for photocosmetic treatments that has a housing, a radiation source capable of producing electromagnetic radiation within the housing, a radiation transmissive element for transmitting said radiation from the source to a target region through a distal end of the housing, and a means for protecting the eye from the radiation.

Preferred embodiments of this aspect of the invention may include some of the following additional features. The apparatus may include at least one of a pressure sensor system, an electrical impedance measurement system, an optical contact sensor system, a reflectometer contact system, an optical diffuser system, a electro oculography (EOG) measurement system, or a mechanical pressure sensor system. A pressure sensor system may have a plurality of buttons disposed around the surface of said radiation transmissive element. The plurality of buttons may project at least about 1 mm from the surface of the housing surrounding the radiation transmissive element. The distal end of the housing may be capable of producing vibrations that have, for example, a frequency in the range of about 100 to about 400 Hz. The device can be a handheld device suitable for use by a consumer.

Another aspect of the invention is a method of protecting an eye during a cosmetic treatment with electromagnetic radiation, comprising: applying a photocosmetic device to a skin tissue; determining whether the skin tissue is associated with the eye; and causing a source of electromagnetic radiation of the photocosmetic device to remain unacctuated if the skin tissue is associated with the eye and to be actuated if the skin tissue is not associated with the eye.

Preferred embodiments of this aspect of the invention may include some of the following additional features. The determination may be based on sensor data from the photocosmetic device and may further comprise compressing a portion of the photocosmetic device against the skin and determining whether the force of the compression exceeds a predetermined threshold. The method may further include measuring a melanin optical density of the skin tissue, and setting treatment parameters based on the measurement. The step of determining whether the tissue is associated with the eye is based on input from the user, for example, based on a sensation of discomfort wherein the user does not engage the device due to the level of discomfort.

Another aspect of the invention is a method of protecting an eye during a cosmetic treatment with electromagnetic radiation, comprising: irradiating a skin tissue with a pulse of electromagnetic energy for measuring a parameter, measuring the parameter based on light from the pulse of electromagnetic energy, using the parameter to determine whether the skin tissue is associated with the eye, and disabling a source of treatment radiation of the photocosmetic device if the skin tissue is associated with the eye.

Preferred embodiments of this aspect of the invention may include some of the following additional features. The parameter may be the level of back reflected light, the spectrum of back reflected light, electrical impedance, or an acoustic signature. The method may also include measuring a melanin optical density of the skin tissue, and setting treatment parameters based on the measurement.

### BRIEF DESCRIPTION OF THE FIGURES

The following drawings are illustrative and are not meant to limit the scope of the invention as encompassed by the claims.

FIG. 1 is a side schematic view of a photocosmetic device.

FIG. 2 is a side perspective view of an interior portion of a photocosmetic device.

FIG. 3 is a side perspective view of a tip of an alternate embodiment of a photocosmetic device.

FIG. 4 is a partially transparent side perspective view of a pin of the photocosmetic device of FIG.

FIG. 5 is a side perspective view of the pin of FIG. 4.

FIG. 6 is a side perspective view of the pin of FIG. 4.

FIG. 7 is a side perspective view of an alternate embodiment of a pin suitable for use in a photocosmetic device.

FIG. 8A is a graph demonstrating the load placed on a set of pins for use in a photocosmetic device when applied to various parts of the face by various persons.

FIG. 8B is a schematic diagram of the locations of the face that were tested as indicated in the graph of FIG. 8A.

FIG. 9A is a schematic diagram of a sensor using optical spectroscopy.

FIG. 9B is a graph of an exemplary reflectance spectra in visible light of facial tissue at various location on the face.

FIG. 9C is a graph of a theoretical reflectance spectra in infrared light of facial tissue at various location on the face.

FIG. 10 is a schematic view of an electrode of an eye-safety mechanism of a photocosmetic device applied to a cross-section of tissue, and also a schematic view of an electrical model of the electrode tissue combination.

FIG. 11 is a schematic view of an alternate embodiment of an eye-safety device utilizing electrical impedance.

FIG. 12 is a schematic view of an alternate embodiment of an eye-safety device utilizing a reflectometer.

FIG. 13 is a graph showing the angular profile of EMR for a reflectometer sensor that is not in complete contact with the skin tissue.

FIG. 14 is a graph showing the angular profile of EMR for a reflectometer sensor that is in complete contact with the skin tissue.

### DETAILED DESCRIPTION

The following embodiments include devices to improve skin and eye safety, especially in the context of photocosmetic devices for use by a consumer.

Although much of the literature concerning eye safety is devoted to the prevention of damage to the retina of the eye, this consideration alone does not ensure complete eye safety. The inventors have discovered that, when performing many dermatological treatments such as, e.g., hair removal and permanent hair reduction, the iris is also vulnerable to damage, in some cases even more so than other parts of the eye, including the retina. This is due to several factors. For example, the iris is composed of a high percentage of melanin, which may selectively and/or preferentially absorb the same wavelengths that are being emitted by the device during treatment of the tissue. Further, for some treatments, the iris lies approximately at a depth below the eyelid that is similar to the depth being targeted in the skin tissue, such as hair follicles.

If a user were to either mistakenly or intentionally place the optical system in contact with his/her eyelid and attempt to fire the laser, depending on the laser power and pulsewidth, it may be possible to thermally damage the iris if the fluence is too high. To prevent the possibility of the laser firing on the closed eyelid, an iris sensor integrated in the handpiece can be used to detect when the optical system is in contact with the eyelid. Therefore, for some photocosmetic and dermatological treatments using electromagnetic radiation (EMR) or other forms of energy, a device will preferably include a mechanism for protecting the iris in addition to other parts of the eye, such as, without limitation, the retina. An effective iris safety sensor can be based on any one of several different technologies, including, without limitation, mechanical sensors, optical spectroscopy, electrical impedance, imaging, and acoustic sensors.

This application discloses novel devices and methods for use in dermatological, cosmetic and other treatments of tissue (primarily skin tissue). The devices and methods include one or more mechanisms and/or procedures for protecting the eye during such treatments. Although the embodiments disclosed herein are primarily intended for use in handheld devices for use by a consumer in a non-medical setting, the principles are applicable to a wide range of devices, including dermatological devices used by a professional and devices used in a medical setting. Furthermore, although the embodiments disclosed herein are primarily optimized for hair removal and/or permanent hair reduction, these embodiments, variations of these embodiments, and other alternative embodiments may be used for other purposes, including, without limitation, treatment of acne, pigmented lesions, vascular lesions, skin rejuvenation, non-ablative skin resurfacing, treatments using fractional technology, wrinkle removal, skin tightening, fat reduction, and the treatment of cellulite. The embodiments disclosed herein irradiate energy generally in the visible and infrared part of the electromagnetic spectrum. However, the principles disclosed are applicable to the broader EMR spectrum as well as other forms of radiant energy such as ultrasound and heat.

### Mechanical Sensors

Since there is no bone immediately behind the eye, it is difficult to press the optical system against the closed eyelid and generate significant pressure. However, in areas where photocosmetic treatments would typically be performed on the face (e.g. upper lip, forehead, chin, etc.), significant pressure can be generated due to the presence of bone and/or muscle below the skin surface. In the case of a square contact tip, arrays of pressure-sensing pins (or contact switches with appropriate pressure thresholds) can be located at the apices of the contact optics of a device. By setting the pressure threshold on the pins to a sufficiently high value, a typical user will not be able to simultaneously exceed the threshold for all four pins by pressing the contact tip against his/her closed eyelid. However, on the intended treatment areas, it is relatively easy for the user to exceed the threshold of the pins.

Other approaches based on the use of pressure sensors are also possible. For example, in addition to the pin pressure sensor, an additional pressure sensor can be used to determine the pressure applied by the user to the handpiece. In this case, when the contact tip is in contact with a treatment area where there is bone just below the skin, the pressure measured by the four pins will be roughly linearly proportional to the pressure applied to the handpiece. However, when the contact tip is in contact with the closed eyelid, the pressure measured by the four pins will not be linearly proportional to the pressure applied to the handpiece due to the soft, conformal nature of the eyelid/eye. The difference in slope (four-pin pressure measurement versus applied handpiece pressure measurement) allows differentiation between skin and eye/eyelid.

One embodiment is a photocosmetic device that includes a pressure sensor that allows the device to deliver EMR only when fully compressed. The pressure sensor allows the tip of the device to be pressed relatively comfortably against the tissue to be treated, but is sufficiently stiff such that it is uncomfortable to press the tip of the device against the eye or a portion of the eye with sufficient pressure to allow the device to deliver EMR. The device preferably is safe and effective in operation on all skin types.

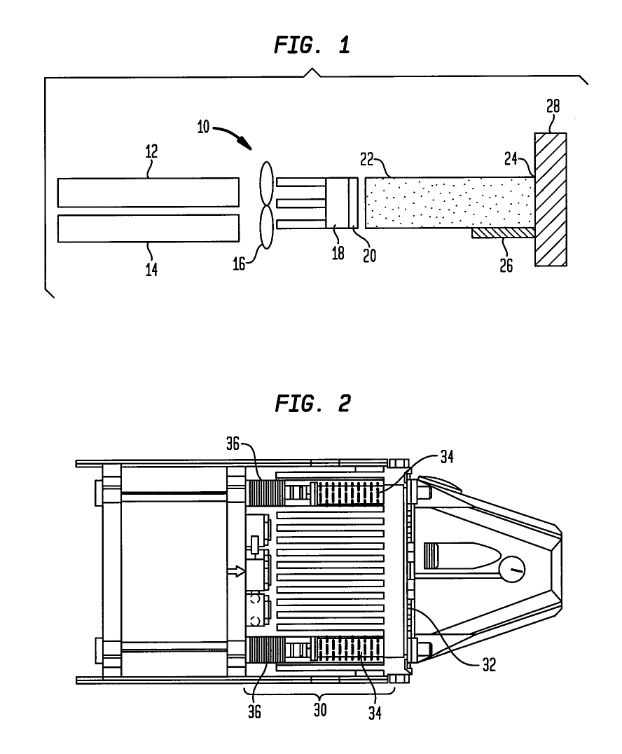

One embodiment of such a device is shown in FIGS. 1 and 2. An exemplary photocosmetic device 10 includes power electronics 12, control electronics 14, a cooling fan 16, a heat sink 18, a source 20 (diode laser), an optical system 22, a contact tip 24 and a cooling dispenser 26. In this embodiment, the EMR source 20 is a source of optical radiation. More particularly, device 10 has a set of three diode bars that produce EMR having a primary wavelength of 980 nm.

Other configurations are possible, and the wavelength may impact the choice of EMR source. For example, a device that produces wavelengths of 800 nm may have two diode bars and include both contact and pigmentation sensors, while, in comparison, a device that produces wavelengths of 1060 mm may have three to four diode bars and include only a contact sensor but omit the pigmentation sensor. These examples are intended only to illustrate preferred configurations, and are not limiting. Many other configurations of EMR sources and sensors are possible.

The contact tip 24 is an 11×11 mm² tip that engages skin 28 during treatment. The skin preferably is moist or has lotion present to ensure optical communication between the optical contact sensor and the skin. In this embodiment, the skin must be in contact over 50% of the tip or the device will not fire. In other embodiments, the device will not fire unless a different percentage of coverage of the tip is achieved, for example, 100% coverage.

A pressure sensor 30 provides eye safety, including protection of both the iris and retina. In this embodiment, the pressure sensor 30 includes a movable head portion 32 that contains the EMR source 20, a set of four springs 34 and a set of four switches 36. In operation, each of the four switches 36 preferably are depressed to allow the device 10 to deliver EMR to the skin 28, and ensure even and full contact with the skin 28 to be treated, and to prevent firing of the device over or into an eye. In this particular embodiment, the springs 34 and switches 36 are disposed at the corners of the interior of the device to effectively use the available space and allow the device 10 to be relatively compact. The springs 34 and switches 36 are disposed about heat sink 18 that extends from and is attached to a surface of the movable head portion 32 to remove heat generated by the EMR source 20. In embodiments with skin cooling, the sensor also improves skin safety by ensuring the there is good contact between the perimeter of the tip and the skin, and thereby providing effective cooling of the skin.

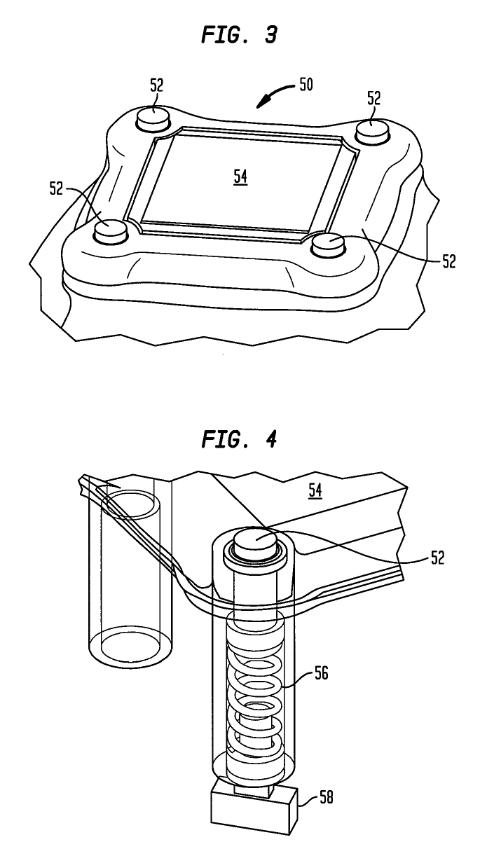

In alternate embodiments, other types of pressure sensors can be use in an eye-safe device. For example, referring to FIGS. 3-6 a device 50 includes a mechanical pressure sensor. The mechanical pressure sensor includes four pins 52 that are arranged about the respective corners of the tip of the device 50. An output window 54, preferably 11×11 mm², is located completely within a perimeter formed by the four pins 52.

While the use of three pins best defines a plane, a non-triangular optic aperture will either fall outside inscribed triangle or be much smaller than the defined triangle. For example, 3 pins could circumscribe an 11 mm square, but the nose footprint would be too large for upper lip and next to nose. Because apertures in photocosmetic devices are generally not triangular, the mechanical pressure sensor preferably has additional pins. However, many other configurations are possible.

The detail of the mechanical pressure sensor is shown in FIG. 4. The mechanical pressure sensor includes a pin 52, a spring 56 and a switch plunger 58. The sensor is disposed in a cavity with a portion of the pin extending through a surface of the tip of the device near the output aperture 54 through which EMR is delivered. The tip of the device is sealed with a Teflon seal. Alternatively, a molded rubber boot that is compliant over the pins can be used to seal the device.

The pins of the mechanical pressure sensor capitalize on the “elastic foundation’ effects of skin. Where the elastic foundation is firmer, less pressure is required to activate the device. Where the elastic foundation is softer, more pressure is required to activate the device. In this embodiment, the device is not based on pain threshold (although alternate embodiments could include such a mechanism). The contact pins are not intended to be noticeable sensation points. More ductile skin and more slack skin will not be able to depress the sensor. For example, the eyelid will conform over and around the pin to the datum (in this case the outer surface of the tip of the device) rather than depressing the pin. In operation, it is very difficult to apply enough pressure to the face to actuate a pin that is over the eyelid.

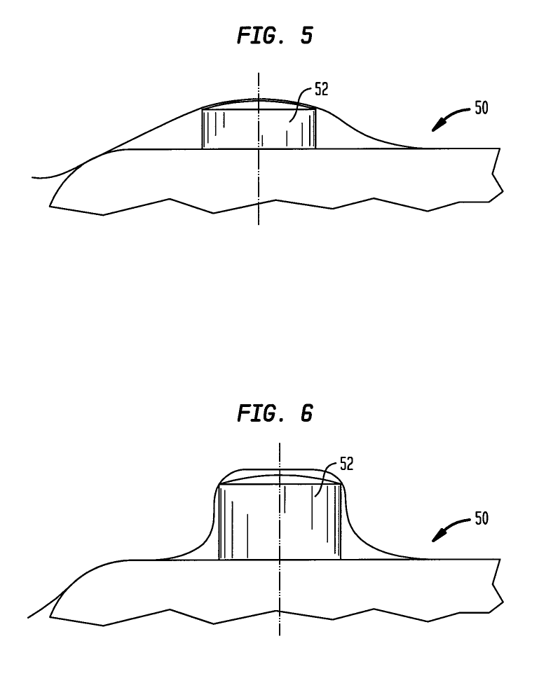

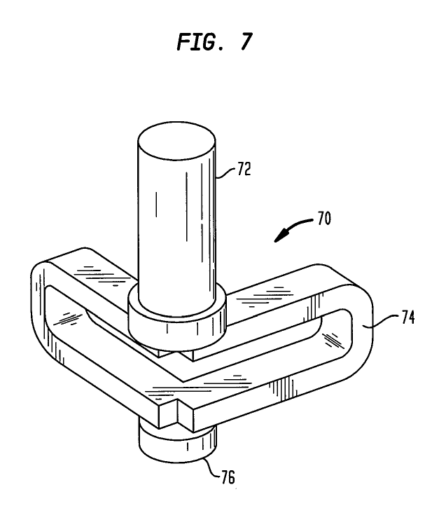

In operation, all four pins 52 preferably are depressed above a threshold value to allow the device to deliver EMR. A tight skin profile (as shown in FIG. 5) results in the pin being depressed above the threshold value when moderate pressure is applied to the device, thus enabling the device to deliver EMR. A slack skin profile (as shown in FIG. 6) results in the pin being depressed only slightly and below the threshold value when moderate pressure is applied to the device, thus enabling the device to deliver EMR. In that case, even increasing the pressure above the comfort level does not cause the pin to be depressed above the threshold value.

Many other configurations and embodiments are possible, however. For example, one alternate embodiment of a mechanical contact sensor for use in an eye-safe photocosmetic device is shown in FIG. 7. The mechanical contact sensor includes a one piece molded plastic element 70 that has a pin section 72, a spring section 74 and a switch engagement section 76. In operation, the pin section 72 extends through the surface of the device near the aperture and the spring section 74 provides the desired resistance. When the pressure applied exceeds the predetermined threshold, the switch engagement section 76 is pressed sufficiently against a switch to engage the switch. In still another embodiment, a mechanical pressure sensor can include a tactile dome switch consisting of a polymer film disposed over a load cell.

In other embodiments, still other types of pressure sensors can be use in an eye-safe device. Such devices include, without limitation, load cells, contact switches and resistive polymer sensors. For example, a load cell can be used that is configured to allow the device to deliver EMR only when the pressure on one or more load cells is above a certain threshold. Preferably, several load cells would be placed about a tip of the device and the device would deliver EMR only when all of the load cells were above a threshold. However, other configurations are possible.

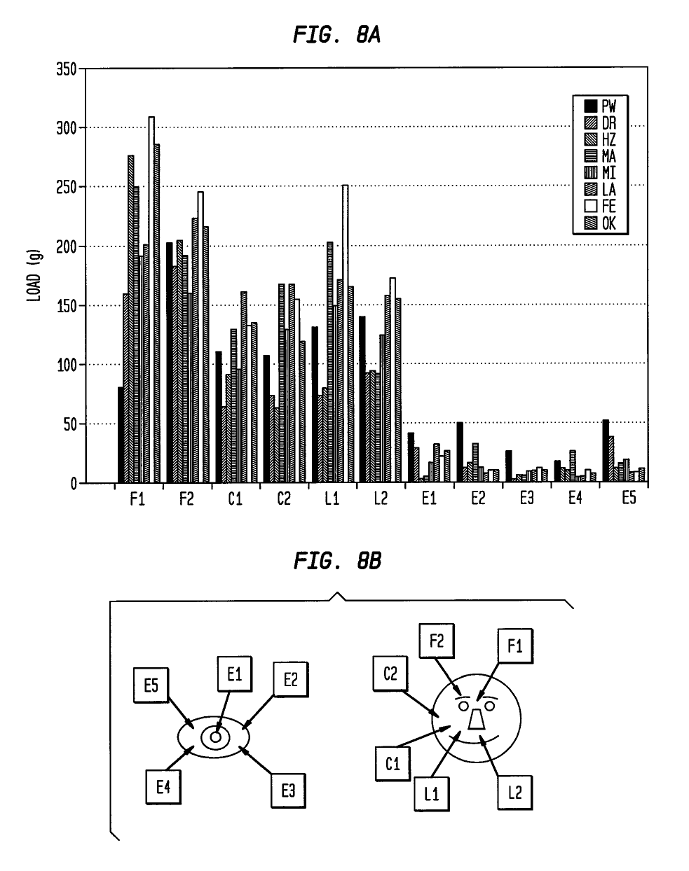

To test the mechanical pressure sensor in device 50, a study was conducted using 8 subjects (five female and three male) to measure the load that can be applied to different parts of the face. The results of the study are shown in FIGS. 8A and 8B. As seen in the graph, the loads that could be applied to various parts of the face (bridge of nose (F1), brow (F2), upper cheek (C1), lower cheek (C2), upper outer lip (L1) and cupid's bow (L2)) were consistently above the loads that could be applied on or around the eye (E1-E5). As can be seen from the graph, the resistance of the mechanical pressure sensor can be set such that the sensor engages above a threshold corresponding to approximately 60 g, as illustrated on the chart.

Additionally, a clinical test was run using 10 subjects (seven female and three male) to determine the success rate for using the device on various parts of the face. The pin diameter of the device used in the test was 2 mm ad the pin height was 1.5 mm. The threshold load per pin was 10 g, which applicants believe to be a relatively low threshold for use with a photocosmetic device intended for consumer use that will make the device easier to use but that may also allow for a slightly higher success rate when attempting to fire the device over the eyelid. The load threshold for all four pins was required to be exceeded in order for the device to engage.

During the study, the operating principle of the pressure sensor prototype was explained to the subjects. The subjects were told they could practice with the device for up to 10 minutes. The subjects then attempted to engage the sensor on several parts of the face: forehead, upper cheek, lower cheek, chin, and upper lip. The subjects could use a hand mirror if they chose to do so. An application was deemed successful if the sensor was satisfied within 2.5 seconds of skin contact. The subjects were then told to perform 10 applications on the right eyelid followed by 10 applications on the left eyelid. The eye application also was deemed successful if the sensor was satisfied within 2.5 seconds of skin contact.

The results of the study are shown in Table 1 below.

TABLE 1
Results of Clinical Study

| | Avg. Value | Avg. Value | Avg. Value |
|---|---|---|---|
| Parameter | 7 females | 3 males | 10 subjects |
| Practice Time | 3.9 minutes | 3.7 minutes | 3.8 minutes |
| Forehead Success Rate | 92.9% | 58.3% | 82.5% |
| Upper Cheek Success Rate | 60.7% | 66.7% | 62.5% |
| Lower Cheek Success Rate | 96.4% | 91.7% | 95.0% |
| Chin Success Rate | 85.7% | 83.3% | 85.0% |
| Upper Lip Success Rate | 85.7% | 75.0% | 82.5% |
| Total Success Rate | 84.3% | 75.0% | 81.5% |
| (5 facial areas) | | | |
| Right Eyelid Success Rate | 1.4% | 13.3% | 5.0% |
| Left Eyelid Success Rate | 1.4% | 0.0% | 1.0% |
| Total Eyelid Success Rate | 1.4% | 6.7% | 3.0% |

The various studies performed using the mechanical pressure sensors illustrated several design considerations. The handpiece should be designed ergonomically to help the user easily keep the contact window near normal to skin, and allow the user to apply reasonable force to exceed load threshold on all four pins. The pin configuration of 2 mm diameter works well for hair removal applications. If the pin is too large it will produce a larger face footprint, but if too small, it may produce an uncomfortable skin sensation. A dome configuration for the pin contact surface works well and is comfortable, but other configurations are possible. The pin height is preferably at least 1 mm with 1.5 mm being a preferred height for many applications. However, other configurations are possible, including less than 1 mm. The spring rate can be varied. A very stiff spring rate may be difficult to use, however. A spring rate of about 11 lb/in works well in many applications. If the spring rate is too high, the elastic foundation effect is not being used effectively and, if the spring rate is too low, more ductile skin will allow actuation. It may be preferable to provide feedback to the user as to the number of pins that have been satisfied, e.g., LEDs or sound, such as tone indicating all satisfied, or alternatively series of pitches progressively higher as each pin threshold is satisfied.

### Optical Spectroscopy

Although pressure sensors as described above are an effective mechanism to protect the eye, including both the iris and the retina, other approaches are possible. For example, a photocosmetic device may instead or additionally include an optical spectroscopy sensor.

An optical spectroscopy sensor can be configured to, for example, employ multiple wavelengths having differential reflection and transmission properties. Thus, when the device is placed over the eye (or a portion of the eye), the reflection and transmission properties of the various wavelengths will be different than when the device is placed over the tissue to be treated. The sensor measures this difference, and sends a signal to a controller that allows the device to deliver EMR only when it detects that the device is over the tissue to be treated and not over the eye or a portion of the eye. One concept for such a sensor is based on the back reflectance measurements from the skin in the visible and/or near infrared ranges. The device measures the changes of backscattering caused by structural specificity of the eye ball, which has highly scattering scleral tissue, a highly absorptive iris tissue that contains melanin and blood, and a high level of hydration of the eye compartment. These components are located behind the eyelid, which is a thin tissue layer that consists of skin and conjunctiva.

By using a light source with a properly chosen wavelength (or wavelengths) and a photodetector to detect backscattered/back reflected light, it is possible to detect when the optical system is in contact with the eyelid. Since the eyeball contains a large amount of water, near-infrared wavelengths near water absorption peaks (e.g. near 1300 nm) are particularly promising.

Although many different combinations of visible and near infrared wavelengths can be considered for the light source, one possible combination is 1200 and 1300 nm. The reflectance at 1200 and 1300 nm is similar for the eyelid over iris and sclera but different for skin.

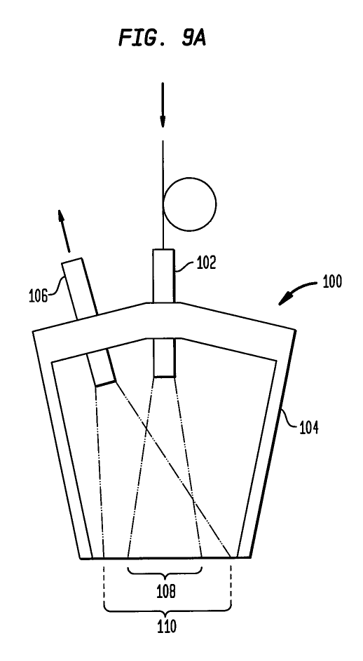

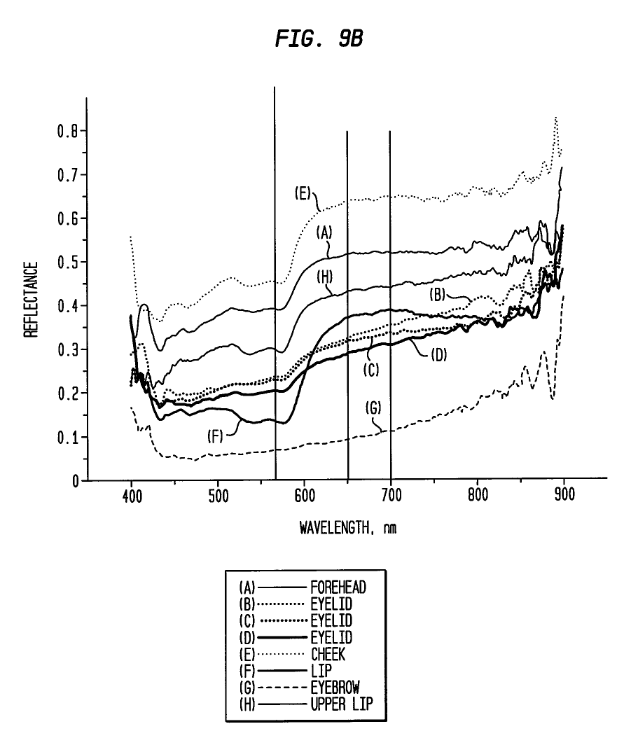

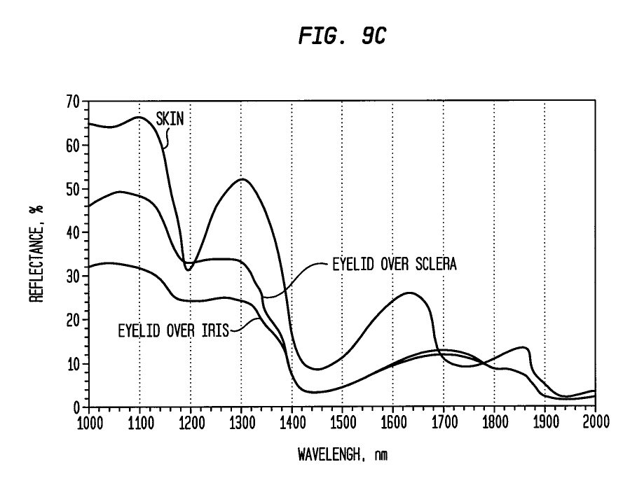

An example of an optical reflectance sensor 100 is shown in FIG. 9. A reflectance sensor 100 includes a source of sensor radiation 102, which is different that the treatment radiation in this embodiment, an optical system 104 and a reflectometer probe 106. The source 102 is a central fiber that emits EMR on a surface of the skin. The EMR is provided by a 200 W-halogen lamp (not shown).

The reflectometer probe 106 preferably collects the reflected radiation from a relatively large area, which is approximately two-fold larger than the irradiated area in this embodiment. The relatively larger area improves the accuracy of the device by collecting almost all back-scattered light, and reduces the chances of misreading data due to the probe geometry. However, other relative sizes and other configurations are possible. The spot size 108 produced by the source is approximately 4 mm. The reflectometer probe 106 in this embodiment is a fiber probe that detects reflected EMR from an area 110 having a diameter of 8 mm. The probe is placed at an angle of less than 150 degrees relative to the longitudinal axis of the central fiber.

In some embodiments, the reflectometer probe 106 also measures skin pigmentation. Depending on the pigmentation measures, one of two parameter sets is used in the present embodiment: one for lighter skin and one for darker skin. However, in other embodiments, many different configurations are possible, including, for example, a separate set of parameters for each skin type. Alternatively, the pigmentation sensor could be a different component, for example, an infrared temperature sensor that measures the amount of heat absorbed by the tissue.

In operation, the central fiber irradiates the skin surface. The reflectometer probe detects the illumination of different areas of the human skin surface. A controller (not pictured) analyzes the information gathered by the probe and determines whether the device is allowed to deliver EMR based on expected thresholds.

The reflectance spectra of the EMR on various facial sites was recorded using an Ocean Optics USB2000 spectrometer (400-900 nm), and is shown in FIG. 9B. As illustrated in the graph, the spectral output at the wavelengths 580 nm, 650 and 700 nm (marked by three vertical lines) has a steeper slope for the eyelid projection of the skin surface in contrast to other skin sites of the face. Additionally the theoretical reflectance data is illustrated for the near infrared range in FIG. 9C. As illustrated in the graph of FIG. 9C, the spectral wavelengths in the NIR spectral range, where water bands are significant, such as 1180, 1450, 1900, are thought to be preferable, due to the high hydration in the front eye chamber (aqueous anterior chamber), which is filled up by a water-like media—aqueous humor. Embodiments of a reflectance sensor can have many configurations, but it may be preferable to include multiple wavelengths, e.g., in both the visible and infrared spectra, to more accurately and consistently determine the skin type and/or location of the device.

Another exemplary embodiment of an eye safe device is a photocosmetic device with a reflectometer and contact sensor. The reflectometer and contact sensor combination ensures that the device is in full contact with skin or the source is disabled and the device cannot emit EMR, while providing additional skin safety by providing a measure of melanin optical density.

For the contact sensor, even a slight distance between the device and the skin will be enough to ensure that the device will not emit EMR. This embodiment is useful both as a contact sensor and as an eye safety sensor, because the skin on the eye is thin and the eye is contoured due both to the roundness of the eye itself as well as the contour of the bone around the eye socket. Thus, it is unlikely that complete contact can be achieved across the tip of the device when attempting to treat around the eye.

An example of such a device 150 is shown in FIG. 12. An optical system 152 of device 150 is illustrated in FIG. 12. The optical system 152 includes a waveguide portion 154, a sapphire aperture 156, an emitter fiber 158, a contact sensor fiber 160 and a reflectometer sensor fiber 162. Each of the fibers extends from the optical system 152 to the skin tissue 164, such that the aperture156, and distal ends of the three fibers 158, 160 and 162 are in contact with the skin tissue 164 during operation. (The reflectometer sensor fiber 162 may be omitted if the contact sensor fiber 160 has sufficient information.) In this embodiment, the emitter fiber is optically connected to an SMD tricolor LED capable of producing EMR at wavelengths of 640, 700 and 910 nm to be detected by the sensors. Each fiber is a plastic fiber having a diameter of 1 mm and a numerical aperture (NA) of 0.5. The device also includes at least one SMD detector in optical communication with the reflectometer fiber 162 (e.g., TSL12T, TSL13T or TSL257T).

The optical system 152 couples light emitted by the diode laser to the skin. The reflectometer sensor fiber 162 may utilize an integrated design and dual-wavelength light source similar to those described in US patent application US 2007/0049910 A1. Many other configurations, parameters and combinations of components are possible. For example, an array of reflectometer fibers can be arranged about the periphery of the aperture.

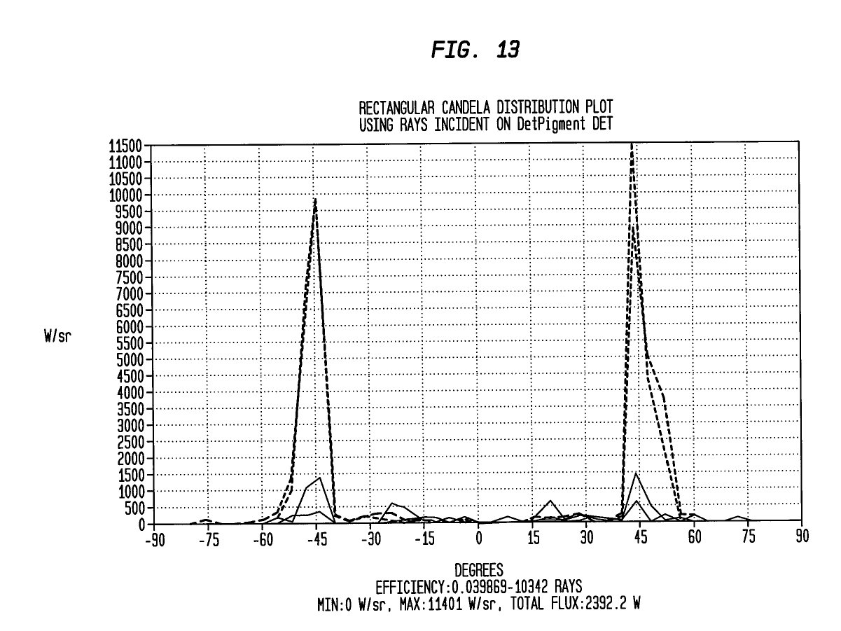

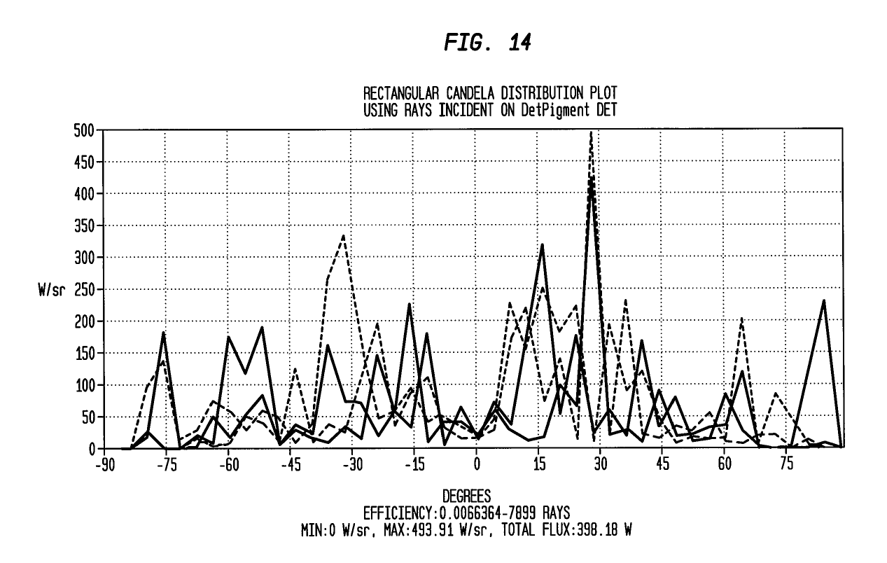

In operation, the aperture 156 of the device is placed near the skin 164. When the device 150 does not touch the skin fully or is even slightly removed from the skin, the device 150 cannot emit EMR. This is due to the angular reflection of the light. The emitter fiber 158 emits EMR for sensing purposes prior to the pulse of treatment EMR. If there is incomplete contact, including a slight air gap, the device 150 will not operate. If there is complete contact and no air gap, the device will operate. As seen by comparing FIGS. 13 and 14, the angular profile of the radiation changes when the device is place on the skin. As shown in FIG. 13, the angular profile of radiation on the reflectometer sensor with the aperture on type II skin with a 1 mm air gap is nearly completely attenuated to zero except at plus and minus 45 degrees. In comparison, as shown in FIG. 14, the angular profile of radiation on the reflectometer sensor with sapphire on type II skin is no longer attenuated. The ability of the reflectometer to detect the change in the angular profile allows the reflectometer to determine that the device is completely in contact with skin tissue.

While the above embodiment is advantageous due to the relatively simple and reliable sensor configuration, one disadvantage of the reflectometer sensor is that small heavily pigmented skin features such as moles (or dark tattoo ink) located within the reflectometer aperture cannot be individually detected because light returning from the skin is incident on a single photodetector. Instead, the overall value of the melanin detected in a given area is averaged over the entire area and the mole or other lesion could be over treated or the level of EMR applied otherwise may not be optimum.

There are several alternate embodiments that address this disadvantage. For example, a relatively small area can be sampled and multiple smaller areas could be sampled and analyzed independently by the control electronics. Alternatively, a large area could be examined to check for an overall average while one or more smaller areas could be sampled to check for deviations from the average and the treatment parameters selected accordingly.

In still another embodiment, light returning from the skin can be imaged onto a CCD camera that is used to detect small heavily pigmented features within the reflectometer aperture. A CCD camera can be mounted near the contact tip to image the eyelid/eye socket to prevent the light source from firing when the contact tip is on the closed eyelid. Multiple visible-wavelength LEDs can be used to provide a fixed level of illumination for the CCD camera. The images from the CCD camera can be processed in software to determine whether the contact tip is on the eyelid. For example, by dividing the image into 1 mm² zones within a 3×10 mm² reflectometer aperture and processing the time-multiplexed image at each of the reflectometer wavelengths, the pigmentation level of each 1 mm² zone can be calculated. Since the reflectometer adjusts the light fluence delivered to the subject's skin based on pigmentation measurements, damage to small heavily pigmented skin features can be avoided by setting the light fluence below the damage threshold of the 1 mm² zone with the highest calculated pigmentation.

### Electrical Impedance Sensors

Another exemplary embodiment of an eye safety sensor is an electrical impedance sensor. By performing AC impedance measurements across a wide range of different frequencies, it is possible to discriminate skin from closed eyelid. The measurement configuration would use a dual-electrode design; the spacing between the electrodes should be sufficient to ensure that the probe electric field penetrates below the eyelid and into the eyeball. The high water content of the eyeball produces an AC impedance spectrum that has significantly different features than skin.

An electrical impedance sensor measures the impedance at different frequencies to determine whether the device is placed over the eye or a portion of the eye. Because the eye has a different structure than skin tissue and is located essentially recess in the skull (whereas other skin tissue is generally located over bone, the electrical properties of eye tissue are different that those of skin tissue to be treated. Thus, when the device is placed over the eye (or a portion of the eye), the electrical impedance will be different than when the device is placed over the tissue to be treated. The sensor measures this difference, and sends a signal to a controller that allows the device to deliver EMR only when it detects that the device is over the tissue to be treated and not over the eye or a portion of the eye. Other electrical properties can be employed other than electrical impedance or in addition to electrical impedance, such as, for example, a measure of current or voltage from a DC source.

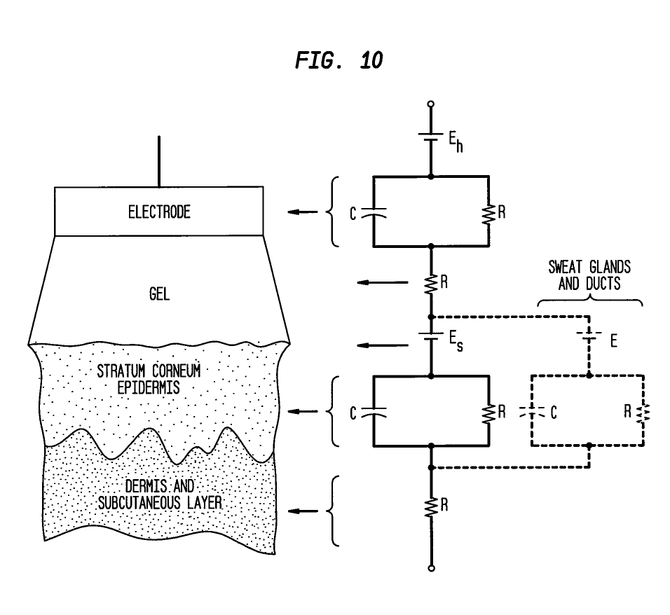

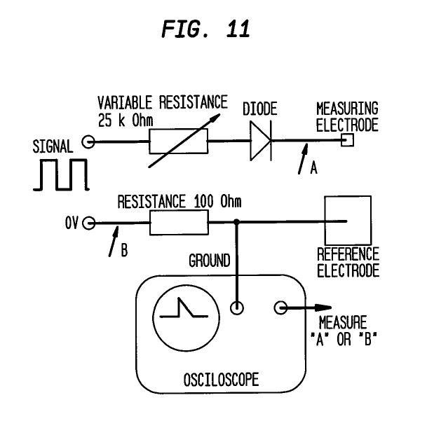

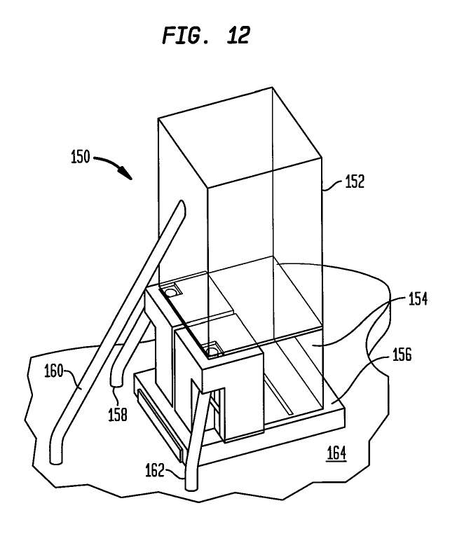

An exemplary electrical impedance sensor is shown in FIGS. 10 and 11. FIG. 10 is a graphical representation of an electrical model of the human skin with an electrode placed on the surface of the skin and a layer of gel applied between the electrode and the surface of the skin. The impedance of the tissue is measured to be approximately 200 kΩ at 1 Hz and 200Ω at 1 MHz. The circuitry for an exemplary impedance measurement receiver sub-system is shown in FIG. 11.

### Acoustic Sensors

Acoustic sensors such as ultrasound are diagnostic techniques used by ophthalmologists to image the eyeball and measure the eyeball length. There are several possible methods of using ultrasound to detect when the contact tip is in contact with the closed eyelid. One method is to use ultrasound imaging to detect the presence of the eyeball beneath the eyelid. A second method is to use a pulsed ultrasound source to probe the area below the contact tip. Due to the difference in acoustic impedance between structures in the eye, it is possible to measure the intensity of the returning ultrasound pulse as a function of time and identify peaks that correspond to the lens, back of the retina, etc. This technique (known as A scan ultrasonography) is used by ophthalmologists to measure the length of the eyeball in patients that will undergo cataract surgery and will subsequently require an intraocular lens.

Another exemplary embodiment of an eye-safe device is an acoustic sensor that detects the reflectance of sound, including ultrasound, rather than (or in addition to) light or other electromagnetic radiation. The eye is almost entirely composed of water, and extends deep into the body relative to the depth of treated tissue. Thus, a sound wave emitted from a device will be reflected or echoed in the eye with a different signature than the same wave emitted above other types of skin tissue, such as facial tissue on the cheek or lips. This difference in signature can be used to determine whether all or some portion of the aperture is located above the eye or otherwise in a position to cause damage. The difference will be seen in both the signature of the echo and in the timing that the echo returns to the device.

In one embodiment, a pulse of eye-safe sound, such as ultrasound or other sound, is emitted from the device. The sound is emitted from various locations about the perimeter of the aperture. The echo is measured by one or more sensors located on the device. A microprocessor or other electronic device compares the received signal against the expected signal allowed for the treatment of tissue. If the comparison determines that one or more of the signals do not correspond to an eye-safe signal, the device is not fired. If the comparison determines that the received signal(s) do correspond to an eye-safe signal, the device is allowed to fire.

In addition to functioning as an eye-safety sensor to determine whether the device is in a position that may damage a portion of the eye, the device can also serve as a contact sensor. For example, the echoed signal that is received when the device is not in contact with any tissue will be different still from the signal received when the device is either in contact with treatable tissue or is in contact with the eyelid.

### Other Eye-Safety Devices and Sensors

Another exemplary embodiment is an electro-oculography sensor (EOG sensor). An EOG sensor measures voltage resulting from the mechanical or optical stimulation of the eye. If the device measures a sufficiently large response following the stimulation of the eye, the sensor sends a signal to a controller that allows the device to deliver EMR only when it detects that the device is over the tissue to be treated and not over the eye or a portion of the eye.

Mechanical or bright light stimulation of an eye will generate its movement, resulting in a proportional potential signal change, which can be detected. A typical voltage will be approximately 10-30 mV. Vertical movements of the eye are preferably measured with electrodes placed on the eye lids. Horizontal movements of the eye are preferably measured with electrodes on the external side of the eye. A photocosmetic device may include integrated electrodes in a handheld unit that stimulate the eye prior to engaging the source of EMR, and prevent engagement if the system detects that the aperture overlaps all or a portion of the eye by detecting the electrical signal from the eye following stimulation. Preferably, the device will have sufficient electrodes configured to measure both horizontal and vertical eye movements, e.g., by placing four sensors around the aperture of the device.

Another exemplary embodiment of an eye safe device is a device having a tip that vibrates. The vibration discourages use of the photocosmetic device on the eyelid by using a vibrating contact tip. The rate of vibration is chosen to provide an uncomfortable sensation when placed against tissue that is unsafe for treatment, such as the eye-lid or the eye itself, while being neutral or pleasant when placed against tissue that is safe for treatment. An exemplary device operates at 200 Hz with a peak-to-peak amplitude of 2V and a triangular waveform. The entire handpiece can be vibrated or only portions, such as the tip of the handpiece, the aperture, or the pins. Embodiments preferably select parameters that discourage use on the eye while allowing use on the upper lip. In some configurations and on some persons, the sensitivity of the upper lip is similar to that of the eyes, and the parameters used should ensure that the sensations are sufficiently distinct. Alternatively, the vibration can be employed for applications that are not to be used on the lip, e.g., hair removal from legs.

An alternate embodiment of the photocosmetic device 10 of FIG. 1 employs a method of cooling both the aperture of the device and the skin. The diode laser is soldered to a finned heat sink, and the fan is used to cool the heat sink. In FIG. 1, the fan is oversized relative to the heat sink in order to provide airflow direct to skin for the purpose of cooling the skin. Proper skin cooling is used in order to deliver the highest possible light fluence to skin and minimize discomfort during treatment. Skin cooling can be enhanced by applying an evaporative lotion (e.g. an alcohol-based lotion or other volatile-liquid based lotion) to the skin. When airflow from the fan is incident on skin where the evaporative lotion has been applied, significant superficial skin cooling will occur as the lotion rapidly evaporates.

Lotion at room temperature can be applied by hand to an area of tissue to be treated such as facial tissue. For example, lotion can be applied to an area of approximately 10 cm². Subsequently, the applicator window can be placed in contact with the skin on the lotion-wetted area.

The sapphire window provides an amount of “stored” cooling from periodic contact with the cooling lotion. This potential is imparted to the wetted window by evaporative (or other) cooling while in air (off skin). During skin contact, the cooled window imparts this stored cooling to the skin.

In tests using the cooling method described above, an applicator, constructed of thermally insulating foam with attached sapphire window and sensors, was used to measure window temperatures during contact with a cooling lotion on the skin surface. Thermistors, attached to front and back window surfaces respectively were used to record thermal response during window contact with skin. The following window thicknesses were tested: 0.25 mm, 0.63 mm, 1.26 mm, 1.90 mm. During the tests, the device was on for two seconds and off for one second. The application sequence was maintained (manually), uninterrupted for approximately 60 seconds over the lotion wetted area. Effort was made to avoid consecutively stamping the same area.

Temperature data was recorded from both thermistors by means of a data acquisition system. Tests were done with and without lotion in order to observe cooling effect of lotion.

Since the window contact area, material properties, and local heat transfer coefficients were constant for all cases, the only variable contributing to thermal storage was the thickness of the optical window at the aperture. Ideally, the window heat capacitance should be low enough to maximize the falling temperature excursion of the window during the OFF cycle (air exposure). This would provide the lowest initial window temperature at the instant of skin contact. If the capacitance is too low however the window will warm up too quickly during ON cycle (skin contact) before imparting sufficient cooling to skin. The criteria were evaluated by observing response of the front and back window temperature sensors respectively.

For this application, the 0.63 mm window provided optimal combination of heat capacitance and relaxation temperature. The results of the tests are shown in Table 2 below.

TABLE 2
Optical Window Cooling Test Results

| | 0.25 mm | 0.63 mm | 1.26 mm | 1.9 mm |
|---|---|---|---|---|
| J/stamp | 0.106 | 0.196 | 0.226 | 0.489 |
| T_avg-T_amb | 4.9 | 3.5 | 4.8 | 4.4 |

The simplest method of spreading the lotion on the skin is to have the user apply it to the treatment areas by hand. Another method is to use a coolant dispenser built in into the handpiece as shown in FIG. 1. An electrical or optical proximity sensor can be used to determine when the contact tip is in close proximity to the skin; a burst of coolant (either liquid or spray form) can be directed at the skin prior to the contact tip touching the skin. This allows pre-cooling of the skin prior to laser irradiation. Alternatively, a series of parallel grooves can be milled across the face of the contact optic, and the evaporative lotion can be pumped through these grooves when the contact tip is in contact with skin. Instead of a separate coolant dispenser, it is also possible to use either a porous contact optic or a contact optic with holes drilled in it to allow delivery of the evaporative lotion to skin. In another embodiment, a lotion with the evaporative component(s) microencapsulated can be spread on the skin by the user. Initially, the lotion does not evaporate. However, when the contact tip presses against the microcapsules, they burst, thereby allowing evaporation to occur. Another method of skin cooling utilizes a chilled hand-held roller that the user rolls across the treatment areas to pre-cool the skin prior to treatment.

In still another embodiment, diffusers, such as holographic light shaping diffusers, can be employed to ensure an eye-safe pulse of EMR. Light shaping diffusers are preferable because their transmission efficiency is superior to that of other types of diffusers, such as plastic diffusers and ground glass. For example, where the transmission efficiency of conventional plastic diffusers and ground glass are on the order of approximately 60-70% for wavelengths greater than 400 nm, the transmission efficiency of light shaping diffusers over the same range of wavelengths is on the order of approximately 90%. These diffusers allow EMR to pass from the device to the tissue when directly adjacent to or in full contact with the tissue, but highly scatter the light when the device is any significant distance from the tissue.

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims. All publications and references cited herein are expressly incorporated herein by reference in their entirety.

1-19. (canceled)

20. A method of protecting an eye during a cosmetic treatment with electromagnetic radiation, comprising:
irradiating a skin tissue with a pulse of electromagnetic energy for measuring a parameter;
measuring the parameter based on light from the pulse of electromagnetic energy;
using the parameter to determine whether the skin tissue is associated with the eye;
disabling a source of treatment radiation of the photocosmetic device if the skin tissue is associated with the eye.

21. The method of claim 20, wherein the parameter is the level of back reflected light.

22. The method of claim 20, wherein the parameter is a spectrum of back reflected light.

23. The method of claim 20, wherein the parameter is electrical impedance.

24. The method of claim 20, wherein the parameter is an acoustic signature.

25. The method of claim 20, further comprising:
measuring a melanin optical density of the skin tissue; and
setting treatment parameters based on the measurement.
